(19) United States
(12) Patent Application Publication
(10) Pub. No.: US 2012/0122728 A1
(43) Pub. Date: May 17, 2012

(54) Method Of Screening Drugs For Reversal Of Amyloid Beta Neurotoxicity

(75) Inventors: James J. Hickman, Orlando, FL (US); Kucku Vaghese, Gainesville, FL (US); Peter Molnar, Szombathely (HU)

(73) Assignee: UNIVERSITY OF CENTRAL FLORIDA RESEARCH FOUNDATION, INC., ORLANDO, FL (US)

(21) Appl. No.: 13/322,911

(22) PCT Filed: May 27, 2010
(86) PCT No.: PCT/US10/36336

§ 371 Date: Feb. 2, 2012

Related U.S. Application Data
(60) Provisional application No. 61/181,718, filed on May 28, 2009.

(51) Int. Cl. C40B 30/06; C12Q 1/02

(52) U.S. Cl. 506/10; 435/29

(57) ABSTRACT

A method of screening a compound for effectiveness in treating amyloid beta neurotoxicity comprises culturing mammalian neurons in serum-free defined medium until the neurons are electrically functional, exposing the electrically stable neurons to amyloid beta, monitoring the exposed neurons for impairment of electrical functionality, and treating the exposed neurons with the candidate drug while monitoring their electrical activity for reversal of impairment. The invention also includes a method of identifying a mammalian neuron having a biological marker conferring predisposition to development of Alzheimer's disease, the method comprising culturing the mammalian neuron in serum-free medium until the neuron is electrically functional, exposing the electrically stable neuron to amyloid beta while monitoring for impairment of electrical functionality as an indicator of presence of said biological marker, and verifying presence of the biological marker by treating the impaired neuron with an anti-amyloidogenic compound while monitoring for return of neuron functionality.

### RELATED APPLICATION

This application claims priority from co-pending provisional application Ser. No. 61/181,718, which was filed on 28 May 2009, and which is incorporated herein by reference in its entirety.

### STATEMENT OF GOVERNMENT RIGHTS

The invention claimed herein was made with at least partial support from the U.S. Government. Accordingly, the government may have certain rights in the invention, as specified by law.

### FIELD OF THE INVENTION

The present invention relates to the field of neurodegenerative diseases and, more particularly, to an in vitro system for screening drugs effective for treatment of nerve cells expressing electrical impairment caused by amyloid beta.

### BACKGROUND OF THE INVENTION

We have demonstrated that high-throughput electrophysiology techniques can be used to measure Amyloid beta (Aβ) toxicity in neurons and that the effects of this toxicity can be reversed by a drug application. In addition, we believe we have identified a new target for drug development for Alzheimer's Disease (AD) that focuses on loss of electrical functionality of the cell that may precede synapse degradation by a considerable period of time. These results support the emerging view that functional impairment of neurons may be more important for the development of AD symptoms than the actual cell death which occurs at later stages of the disease [1,2].

AD is the most common cause of dementia in the elderly [3]. The hallmarks of this disease consist of senile plaques composed of Aβ, neurofibrillary tangles and extensive neuronal degeneration [4]. Aβ is a 39-43 amino acid peptide derived from the cleavage of a larger protein, Amyloid Precursor Protein (APP), and is toxic to neurons in vivo and in vitro [5]. The amyloid cascade hypothesis implicates Aβ as having a crucial role in the pathogenesis of AD [6] and as a result is an important therapeutic target. Recent results have implicated soluble aggregates of Aβ for many of the toxic effects of Aβ described in AD [7].

Although it is well known that one of the early hallmarks of AD is marked synaptic degeneration, its cause is only marginally understood [8,9]. According to the leading theory, soluble oligomers of Aβ have a direct “synaptotoxic” effect at nanomolar concentrations [10,11]. Other authors have emphasized the success of Memantine, an NMDA antagonist, in moderate and severe cases of AD, and claim that excitotoxicity could play a role in synaptic degeneration [12-14]. Our hypothesis is based on the observation that Aβ decreases spontaneous activity of neurons at low concentrations and that this has a deleterious effect on cell functionality without significant cell death. According to established theories, this decreased activity would lead to the automatic elimination of synapses with low activity, but at a later time [15,16].

It has also been shown that Aβ toxicity can be reversed to varying degrees using anti-amyloidogenic compounds (AACs) such as curcumin [17]. Curcumin has been shown to have anti-oxidant and anti-inflammatory properties [18] and reduce amyloid plaque burden in transgenic APPsw mice [19]. More recently, curcumin has been shown to reduce the number of aggregates from monomeric Aβ as well as promote disassembly of preformed Aβ aggregates, in addition to inhibiting Aβ oligomer formation and Aβ toxicity at significantly lower concentrations than Ibuprofen [20].

Most in vitro functional electrophysiological studies on the effects of Aβ on neurons have been carried out using the patch clamp method [2,21,22]. Although the use of this technique enables the acquisition of detailed information concerning Aβ effects at the ion channel level, it is very low throughput and complicated relative to extracellular electrophysiological techniques. A recent technological advance for non-invasive chronic monitoring of neuronal and cardiac cell electrical activity is the use of multielectrode array (MEA) recordings of action potentials [23-27]. In contrast to the more common intracellular electrophysiological techniques which usually enAβle only short term (a few hours) monitoring of the activity of cells, MEAs are ideal for investigating long-term/chronic drug effects and also does not limit the number of cells that can be recorded from, at a single instance [24,26,28-30]. Moreover, because MEAs do not require precise positioning of electrodes, they can be used in high-throughput pharmaceutical screens [31]. The most common applications of MEAs include physiological or pharmacological studies in brain slices and in dissociated cell cultures of electrogenic cells including hippocampal neurons [32,33], spinal cord neurons [34] and cardiac myocytes [24,25], among others. Recent developments in the pharmacological applications of MEA technology [31] have shown that introduction of high-throughput functional in vitro electrophysiological assays in drug development could have significant benefits compared to the traditional in vivo or ex vivo assays. For example, electrophysiologically active in vitro neuronal networks have been maintained on microelectrode arrays for over 9 months [34]. More recently, neurons on MEAs have been used to study various drug effects including antidepressants [35], ACHe inhibitors [36] and Zn toxicity [37].

### SUMMARY OF THE INVENTION

Soluble oligomers of amyloid beta (Aβ) are considered to be one of the major contributing factors to the development of Alzheimer's disease. Most therapeutic development studies have focused on toxicity directly at the synapse.

Patch clamp studies detailed here have demonstrated that soluble Aβ can also cause functional toxicity, namely it inhibits spontaneous firing of hippocampal neurons without significant cell death at low concentrations. This toxicity will eventually lead to the loss of the synapse as well, but may precede this loss by a considerable amount of time. In a key technological advance we have reproduced these results utilizing a fast and simple method based on extracellular electrophysiological recording of the temporal electrical activity of cultured hippocampal neurons using multielectrode arrays (MEAs) at low concentrations of Aβ (1-42). We have also shown that this functional deficit can be reversed through use of curcumin, an inhibitor of Aβ oligomerization, using both analysis methods.

The MEA recording method utilized here is non-invasive, thus long term chronic measurements are possible and it does not require precise positioning of electrodes, thus it is ideal for functional screens. Even more significantly, we believe we have now identified a new target for drug development for AD based on functional toxicity of hippocampal neurons that could treat neurodegenerative diseases prior to the development of mild cognitive impairment.

With the foregoing in mind, the present invention advantageously provides a high-throughput in vitro method for the assessment of Aβ effects on spontaneous activity of cultured neurons which can be adapted for high-throughput pharmaceutical screening. This assertion is supported by the emerging view that functional impairment of neurons might be more important for the development of AD symptoms than the actual cell death which occurs at later stages of the disease [1,2]. The results obtained with MEAs correlate well with those obtained using patch clamp electrophysiology wherein Aβ at low concentrations had a deleterious effect on cell functionality without significant cell death. We have also shown that this effect can be reversed to varying degrees using an anti-amyloidogenic compound. The MEA recording method utilized here is non-invasive, thus long term chronic measurements are possible and it does not require precise positioning of electrodes, thus it is ideal for functional screens. Even more significantly, we believe we have now identified a new target for drug development for AD based on functional toxicity of hippocampal neurons.

Accordingly, the present invention provides a method of screening a candidate drug for effectiveness in reversing amyloid beta neurotoxicity. The method of the invention comprises culturing embryonic rat neurons on DETA-coated microelectrode arrays in serum-free defined medium. The method continues by incubating the culture until the neurons become electrically functional and then contacting the electrically stable neurons with amyloid beta. The method then calls for monitoring the neurons for impairment of electrical functionality following contact with the amyloid beta; and for treating the neurons by contacting with the candidate drug while continuing to monitor for reversal of the electrical impairment.

A slight variation of the method of the invention is a method of screening a compound for effectiveness in treating amyloid beta neurotoxicity. This method comprises culturing mammalian neurons in serum-free defined medium until the neurons are electrically functional; exposing the electrically stable neurons to amyloid beta; monitoring the exposed neurons for impairment of electrical functionality; and treating the exposed neurons with the candidate drug while monitoring their electrical activity for reversal of impairment.

Conversely, the invention also provides a method of identifying a mammalian neuron having a biological marker conferring predisposition to development of Alzheimer's disease. This method comprises culturing the mammalian neuron in serum-free medium until the neuron is electrically functional, then exposing the electrically stable neuron to amyloid beta while monitoring for impairment of electrical functionality as an indicator of presence of said biological marker; and, lastly, verifying presence of the biological marker by treating the impaired neuron with an anti-amyloidogenic compound while monitoring for return of neuron functionality.

The first two methods of the invention have industrial applicability in identifying drug products that show effectiveness against Aβ. The third method of the invention provides an in vitro test by which someone may be diagnosed as having a predisposition to developing Alzheimer's disease.

### BRIEF DESCRIPTION OF THE DRAWINGS

Some of the features, advantages, and benefits of the present invention having been stated, others will become apparent as the description proceeds when taken in conjunction with the accompanying drawings in which:

FIG. 1 shows an immunoblot of Aβ oligomers; from left to right: lane 1 is the monomer; lane 2 indicates the apparent inhibition of Aβ oligomerization in the presence of Curcumin; and lane 3 indicates Aβ oligomers;

FIG. 2 illustrates the effect of 24 h exposure to 100 nM Aβ on cell functionality measured using whole cell patch clamp electrophysiology; changes after exposure to Aβ for 24 hr. in spontaneous firing frequency; ***: p=0.00007 (A), K+ ion channel currents; **: p=0.008 (B), and membrane potential; *: p=0.035 (C). Cell survival after administration of 100 nM Aβ at 24 hours and 7 days; *: p=0.04 (D). Data is presented as mean6SEM; N=30 utilizing a two-sample Student's t-test;

FIG. 3 provides graphs showing the time course of the effect of Aβ on spontaneous activity of cultured embryonic rat hippocampal cells at various concentrations; concentrations of 100 nM and 5 mM Aβ caused a complete cessation of firing activity with different time delays (N=5) (A); composite logarithmic graph of the time taken for cells to stop firing at the various Aβ concentrations tested (B);

FIG. 4 shows Aβ induced cytotoxicity in hippocampal cells on MEAs; cell survival before Aβ treatment (A) after treatment with 100 nM Aβ (B) and 20 uM Aβ (C); green denotes live cells; red denotes dead cells; scale bar: 30 mm; percentage of live cells after treatment with various concentrations of Aβ (D). * p,0.5, **p,0.01, ***p,0.001;

FIG. 5 depicts the time course of the application of 20 uM Aβ on spontaneous activity of cultured embryonic rat hippocampal cells on MEAs; spontaneous firing observed before administration of 20 uM Aβ (A); spontaneous firing observed 45 minutes after administration of 20 uM Aβ (B), 90 minutes after administration of 20 uM Aβ (C) and 150 minutes after administration of 20 uM Aβ (D);

FIG. 6 presents the reversal of the effect of Aβ by curcumin, measured using whole cell patch clamp electrophysiogy; Curcumin was coadminstered with Aβ or sequentially applied after Aβ exposure; the values in FIG. 1 were used for baseline comparison; 100% implies complete reversal of Aβ effect and 0% implies no reversal; effect on firing frequency; ***: p=0.0007 (A); effect on potassium currents; **: p=0.005 (B); effect on membrane potential; ***: p=0.0009, **: p=0.009 (C); Curcumin treated groups were compared with Aβ-only groups using a two-sample Student's t-test; data are presented as mean±SEM;

FIG. 7 shows the reversal of the effect of Aβ on firing frequency by curcumin applied together with Aβ or after Aβ exposure; time course of the effect of curcumin on spontaneous firing frequency of embryonic hippocampal neurons when coadministered with Aβ (N=5) (A); time course of curcumin effect on spontaneous firing frequency of embryonic hippocampal neurons when administered after cells were exposed to Aβ for 24 h (N=5) (B); 100% implies baseline values before exposure to Aβ;

FIG. 8 shows a method of screening a candidate drug for effectiveness in reversing amyloid beta neurotoxicity, the method comprising; and

FIG. 9 is a flow diagram for a method of identifying a mammalian neuron having a biological marker conferring predisposition to development of Alzheimer's disease, according to an embodiment of the present invention.

### DETAILED DESCRIPTION OF THE PREFERRED EMBODIMENT

The present invention will now be described more fully hereinafter with reference to the accompanying drawings, in which preferred embodiments of the invention are shown.

Unless otherwise defined, all technical and scientific terms used herein are intended to have the same meaning as commonly understood in the art to which this invention pertains and at the time of its filing. Although various methods and materials similar or equivalent to those described herein can be used in the practice or testing of the present invention, suitable methods and materials are described below. However, the skilled should understand that the methods and materials used and described are examples and may not the only ones suitable for use in the invention.

Moreover, it should also be understood that due to the inherent variabilities of measurements, any temperature, weight, volume, time interval, pH, salinity, molarity or molality, range, concentration and any other measurements, quantities or numerical expressions given herein are intended to be approximate and not exact or critical figures unless expressly stated to the contrary. Hence, where appropriate to the invention and as understood by those of skill in the art, it is proper to describe the various aspects of the invention using approximate or relative terms and terms of degree commonly employed in patent applications, such as: so dimensioned, about, approximately, substantially, essentially, consisting essentially of, comprising, and effective amount.

Further, any publications, patent applications, patents, and other references mentioned herein are incorporated by reference in their entirety as if they were part of this specification. However, in case of conflict, the present specification, including any definitions, will control. In addition, the materials, methods and examples given are illustrative in nature only and not intended to be limiting.

Accordingly, this invention may be embodied in many different forms and should not be construed as limited to the illustrated embodiments set forth herein. Rather, these illustrated embodiments are provided so that this disclosure will be thorough, complete, and will fully convey the scope of the invention to those skilled in the art. Other features and advantages of the invention will be apparent from the following detailed description, and from the claims.

### Materials and Methods

### Microelectrode Arrays

The MEAs and accompanying accessories, including the temperature controller, stimulator, amplifier and MC_Rack V 3.5.8 data acquisition software were obtained from ALA Scientific (Westbury, N.Y.) and Multichannel Systems (Reutlingen, Germany). The MEAs comprised of a glass base that acted as a substrate, gold connector contacts and electrodes composed of titanium nitride. Rings were made of Sylgard184 (Dow Corning) (1 part curing base and 10 parts elastomer base, cured at 60 uC for 45 minutes) using glass molds and were attached onto the MEAs after surface modification. Recordings were obtained from 12-16 D. old cultures. Cultures were kept in the incubator between recording sessions.

### Surface Modification

N-1(3-[trimethoxysilyl]propyl)-diethylenetriamine (DETA) was used to modify the MEAs to enhance cell attachment since the use of synthetic substrates such as DETA, allows for reproducible and precise quantification of the culture substrate properties [43]. Glass coverslips (18 mm diameter, Number 1; VWR) were cleaned in two steps. First, they were soaked in 50/50% HCl (37%) (VWR)/methanol (Sigma), followed by H2SO4 (98%) (VWR) treatment. Next, they were rinsed in double distilled water. The coverslips were then boiled in deionized water, rinsed with acetone, and oven dried. The MEAs were initially cleaned overnight in 2% Tergazyme (Sigma) detergent solution. They were then rinsed in distilled water and plasma cleaned in a plasma cleaner (Harrick Plasma) for 30 mins. The N-1(3-[trimethoxysilyl]propyl)-diethylenetriamine (DETA) (United Chemical Technologies) surface assembled monolayer (SAM) film was formed by the reaction of the cleaned surfaces with a 0.1% (v/v) mixture of the organosilane in toluene. The DETA/toluene solution containing the MEAs was heated to 70 uC, rinsed with toluene, reheated to 70 uC, and then oven dried. Surfaces were characterized using contact angle measurement and X-ray photoelectron spectroscopy (XPS) as described previously [44].

### Cell Culture

All applied procedures were approved by the Institutional Animal Care and Use Committee of UCF. The protocol was modified from previously published work concerning embryonic rat hippocampal cultures [43,45]. Pregnant rats, 18 days in gestation, obtained from Charles River were euthanized with carbon dioxide and the fetuses were collected in ice cold Hibernate E (BrainBits)/B27/Glutamax™/Antibiotic-Antimycotic (Invitrogen) (dissecting medium). Each fetus was decapitated and the whole brain was transferred to fresh ice cold dissecting medium. After isolation, the hippocampi were collected in a fresh tube of dissecting medium. Hippocampal neurons were obtained by triturating the tissue using a Pasteur pipette. In order to remove any debris from damaged cells the 1 ml cell suspension was layered over a 4 ml step gradient (Optipep diluted 0.505:0.495 (v/v) with the dissecting medium and then made to 15%, 20%, 25% and 35% (v/v) in the dissecting medium) followed by centrifugation for 15 min at 800 g and 4 uC. After centrifugation, one strong band of cells was obtained. This band of cells was resuspended in culture medium (Neurobasal/B27/Glutamax™/Antibiotic-Antimycotic) and plated at a density of 100 cells/mm2 on DETA coated coverslips for patch clamp electrophysiology and at 200 cells/mm2 on the MEAs.

### Aβ (1-42) and Curcumin Administration and Electrophysiology

Different concentrations of Aβ (1-42) (Bachem) aggregates were prepared according to the protocol by Klein [1] in Neurobasal medium without phenol red, and quantified using Immunoblots. Curcumin (Cayman Chemicals) was prepared and the concentration chosen according to previously published protocols [20].

For patch clamp electrophysiology experiments, Aβ was administered to the cells on day 10 in vitro and recordings were performed after 24 hrs to obtain baseline values for control cells and Aβ treated cells. In coadministration experiments, a mixture of 100 nM Aβ and 1 mM curcumin was administered for 24 hrs after which patch clamp electrophysiology recordings were performed. In sequential administration, cells were initially exposed to freshly aggregated Aβ alone, followed by replacement of the Aβ solution with curcumin for 24 hrs patch clamp recordings were performed 24 hrs after curcumin treatment. Whole-cell patch clamp recordings were performed at room temperature in a recording chamber on the stage of a Zeiss Axioscope 2 FS Plus upright microscope as described in [44].

In all experiments with MEAs Aβ was administered to the cells on day 16 in vitro. We chose experimental paradigms similar to those used for patch clamp electrophysiology, to study the potential therapeutic effects of curcumin. In coadministration experiments, cells were exposed to a mixture of 100 nM Aβ and 1 mM Curcumin for 24 hrs, and recordings were performed. In the second paradigm, cells were initially exposed to freshly aggregated Aβ alone, followed by replacement of the Aβ solution with curcumin for 24 hrs. Extracellular recordings were obtained before the administration of Aβ, immediately after Aβ administration, 24 h after Aβ administration, before curcumin administration and 24 hrs after curcumin administration.

### Results

Embryonic rat neurons were plated at a density of 100 cells/mm2 on DETA coated coverslips for patch clamp electrophysiology and at 200 cells/mm2 on DETA coated microelectrode arrays in serum free medium. Patch clamp electrophysiology was performed after 10 days in culture as electrical function of the neurons had stabilized at this point. Sporadic firing could also be detected after 10 days using the MEAs. Starting on day 12 we were able to obtain stable, reliable recordings from the MEAs over a period of two to three days with an average firing frequency of 2.560.6 Hz (mean6SEM). This enabled the study of the time course of the action of low concentrations of Aβ on the neurons. Transferring the MEAs from the incubator to the recording head stage and subsequent media changes did not significantly affect the cells. No significant changes in the baseline recordings from control MEAs were observed as a result of transferring the MEAs from the incubator to the recording headstage or media changes.

### Application of Aβ Abolished Spontaneous Spiking Activity

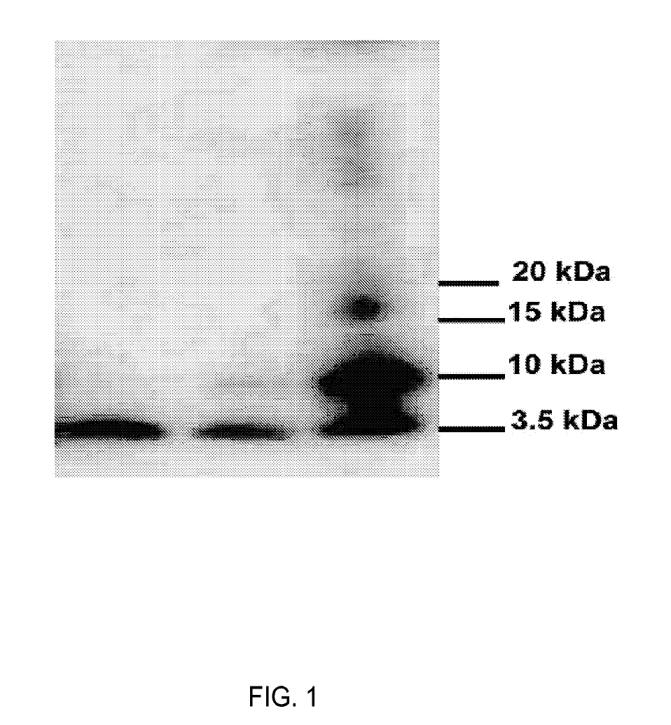

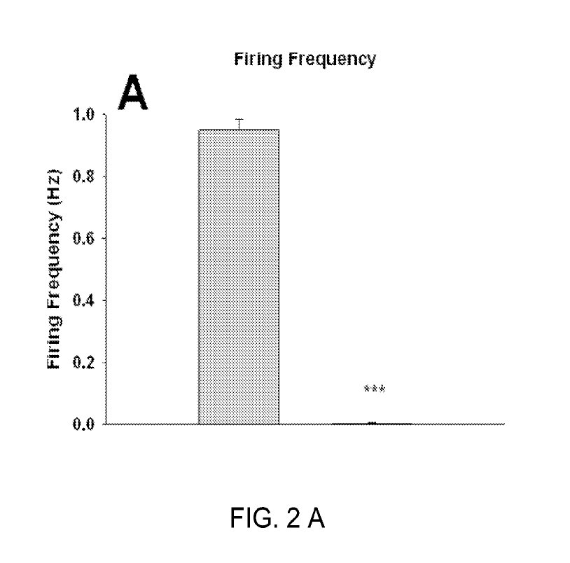

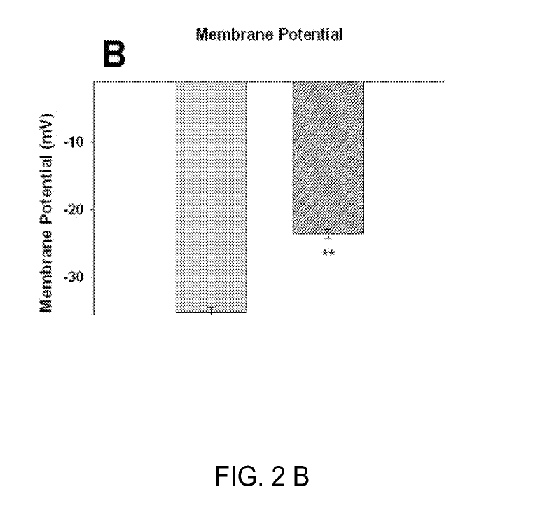

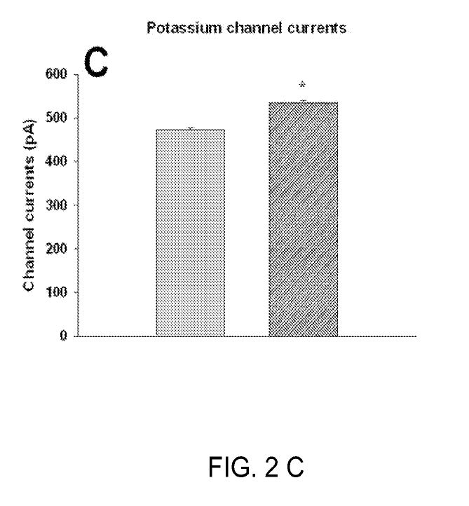

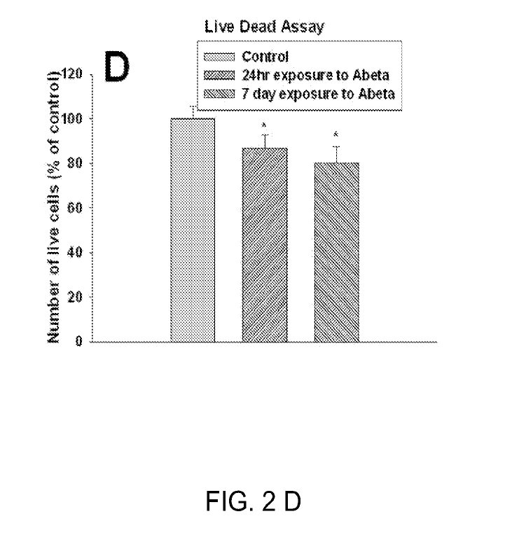

The presence of Aβ oligomers was verified using immunoblots as shown in FIG. 1. Patch clamp experiments performed 24 h post-Aβ exposure revealed striking changes in the neuronal function upon exposure to 100 nM Aβ. The most significant effect was observed on spontaneous firing, namely no spontaneous action potentials were recorded in the 30 exposed cells that were studied at the 24 h time point (FIG. 2A). Exogenous application of Aβ to the cells for 24 h caused an increase in the amplitude of the outward (K⁺) currents as well as a depolarization in the resting membrane potential, (FIG. 2B, C). Given the small differences in cell survival compared to the control, even after 7 days (FIG. 2D), we concluded that loss of electrophysiological function is the major response to Aβ treatment at this concentration. To confirm this finding a MTT assay (3-[4,5-dimethylthiazol-2-yl]-2,5-diphenyl tetrazolium bromide) was performed on the amyloid treated cells and this supported the results obtained with the live dead assay.

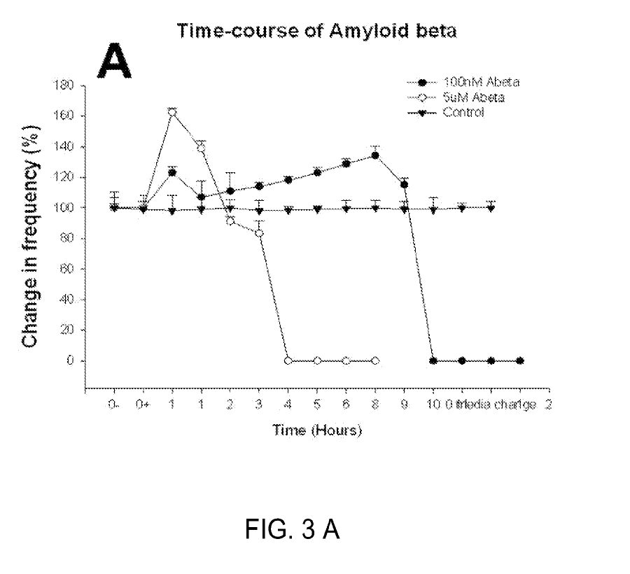

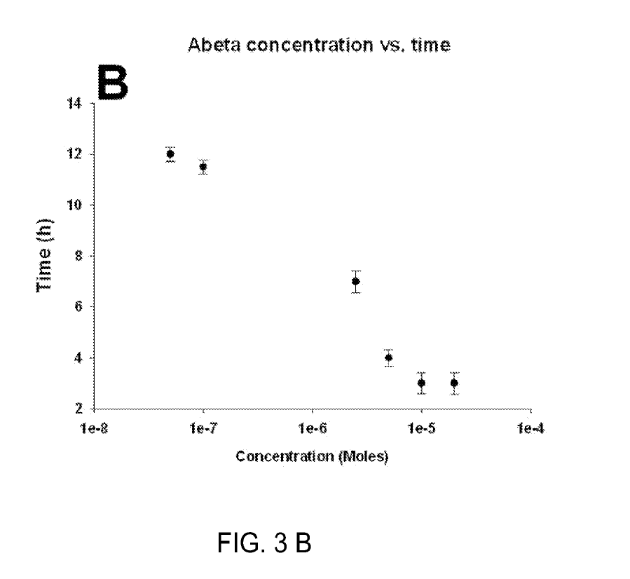

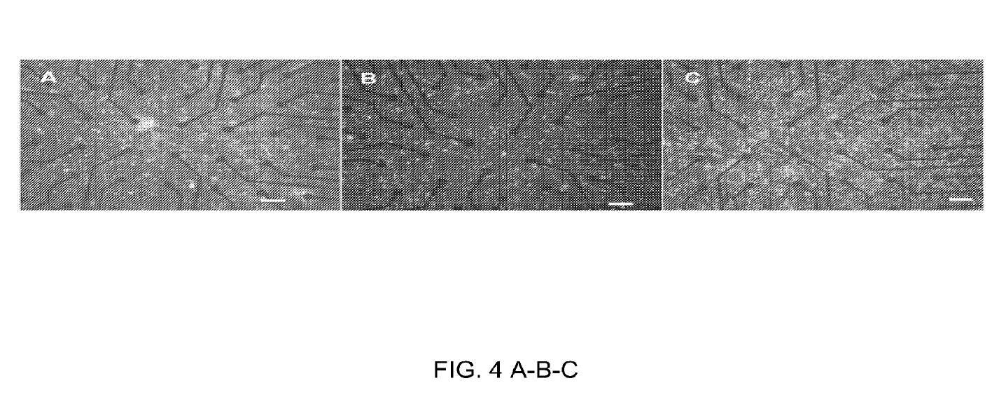

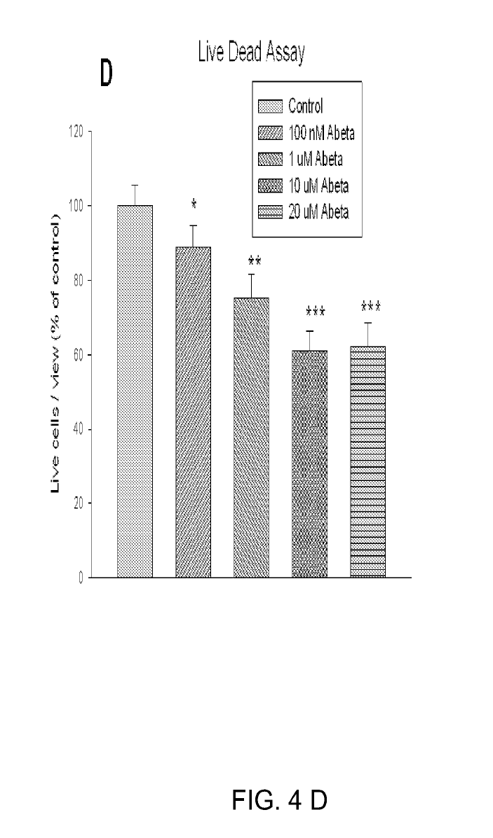

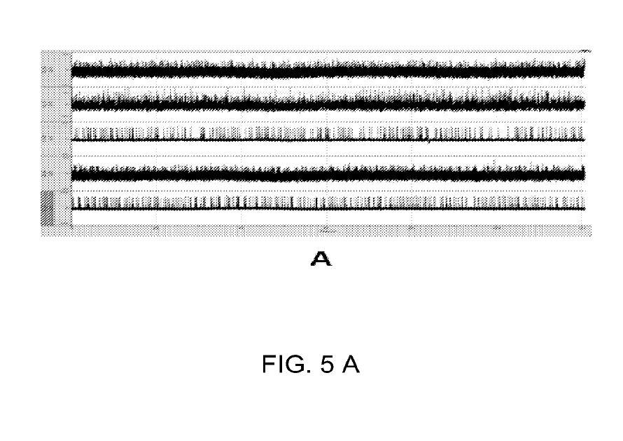

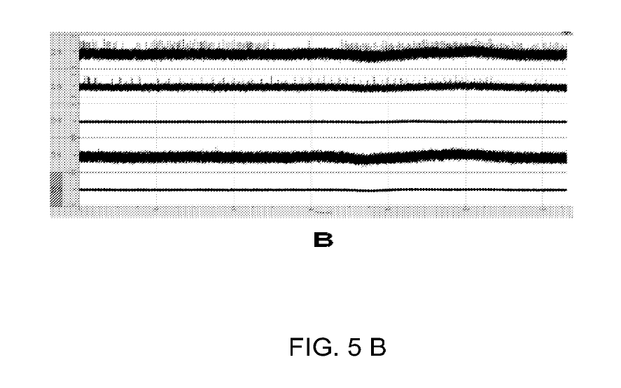

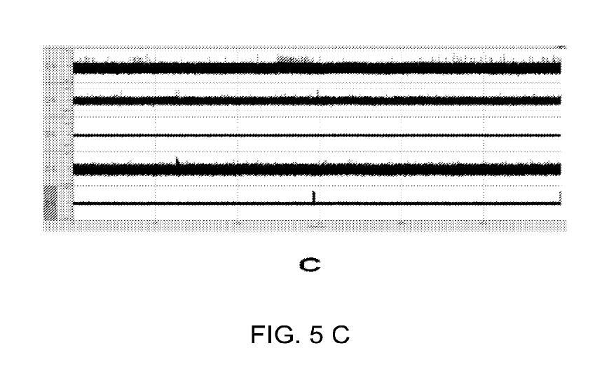

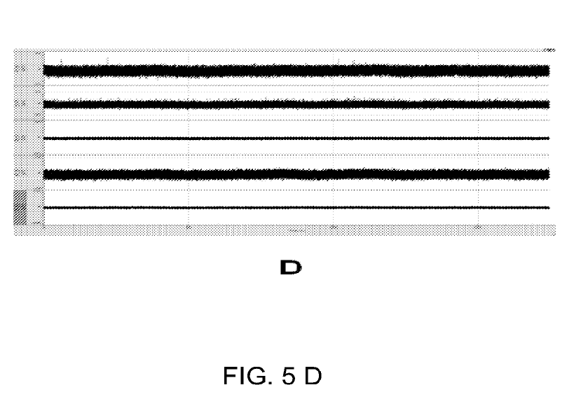

As a result of the significant effect of Aβ on the firing frequency of the hippocampal neurons, it was decided to use this parameter as a possible new target for implementation in high throughput screens utilizing MEAs. The effect of various concentrations of Aβ on the firing frequency of the neurons on the MEAs that were studied is shown in FIGS. 3 and 4. At all measured concentrations, Aβ completely abolishes spontaneous spiking activity whereas application of the vehicle control had no effect (FIG. 3A). The concentration dependence of Aβ action was quantified by measuring the time required for complete blockade of spiking activity as the dependent parameter in contrast to a more traditional concentration/inhibition relationship. When the cells were exposed to 20 mM Aβ, the highest concentration tested, spiking activity of the cells stopped after about 150 min. At the lowest concentration (50 nM) the cells stopped firing after about 11 h. As seen in FIG. 3B, the time for cessation of spike activity reached a plateau at around 10 mM at the higher end of the concentration range. Blockade of spontaneous activity was preceded by a significant increase in firing frequency at all measured concentrations. At high concentrations of Aβ, high cell death was observed as seen in FIG. 4. FIG. 5 shows the time course effect of 20 mM Aβ on spontaneous firing frequency of the embryonic hippocampal neurons. These results were in accordance with those obtained using whole cell patch clamp electrophysiology as indicated in FIG. 1.

### Partial Functional Activity Could Be Recovered by Administration of Curcumin

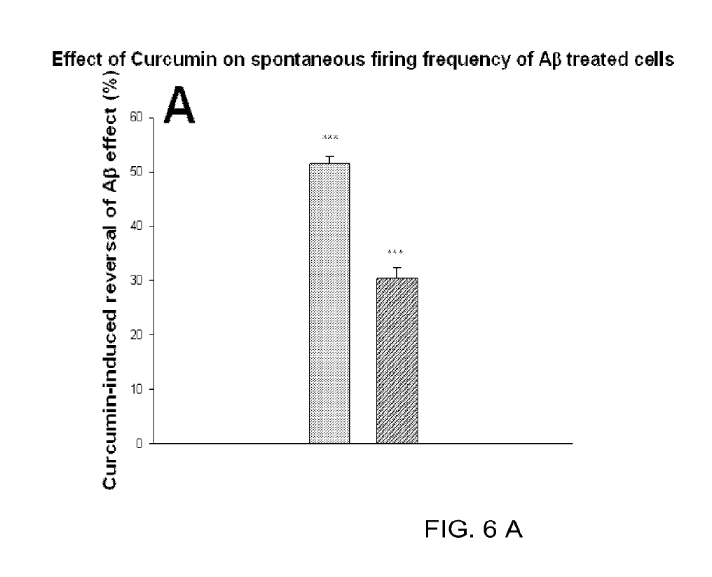

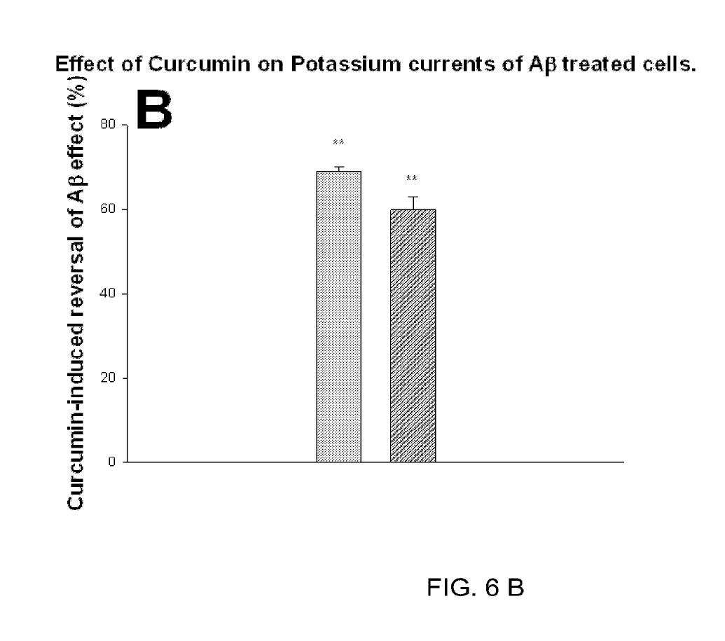

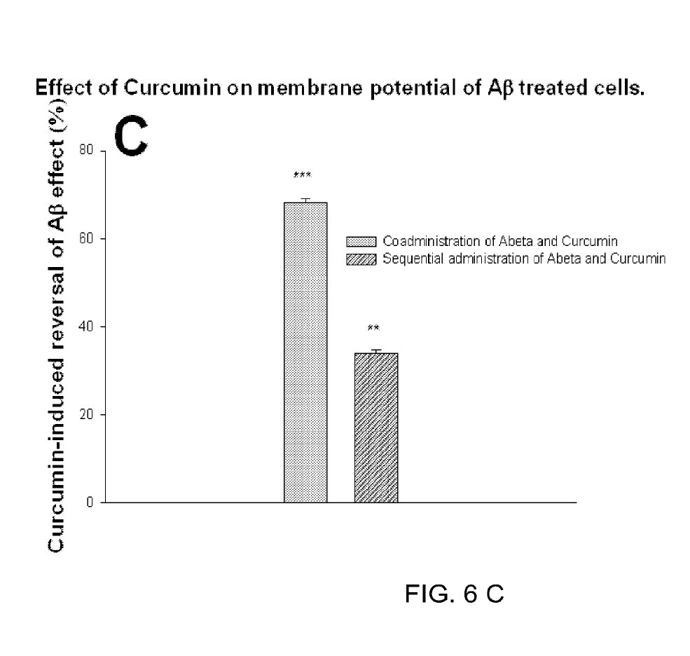

In our patch clamp experiments we determined that low doses of curcumin (as previously published) were more successful in ameliorating Aβ toxicity when coadministered with Aβ as opposed to administration after 24 h exposure to Aβ (FIG. 6).

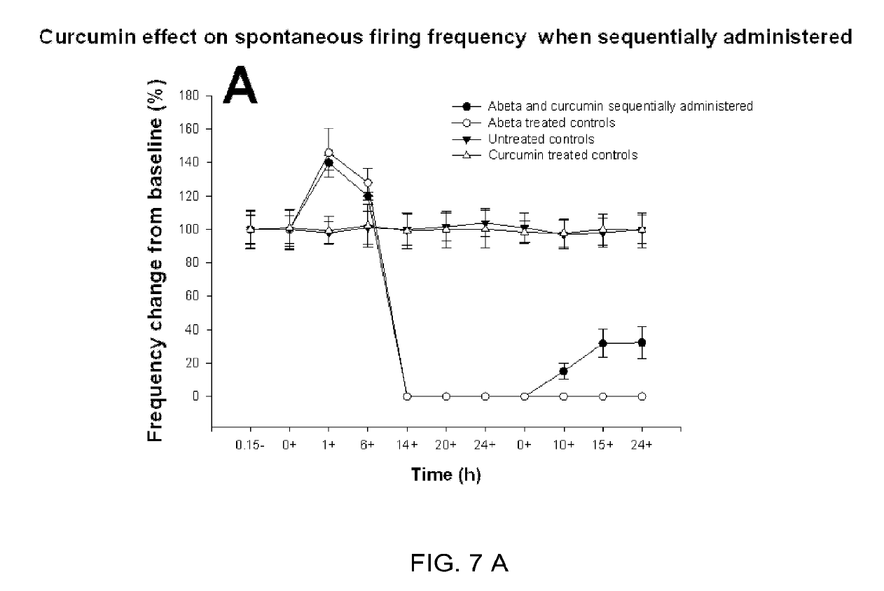

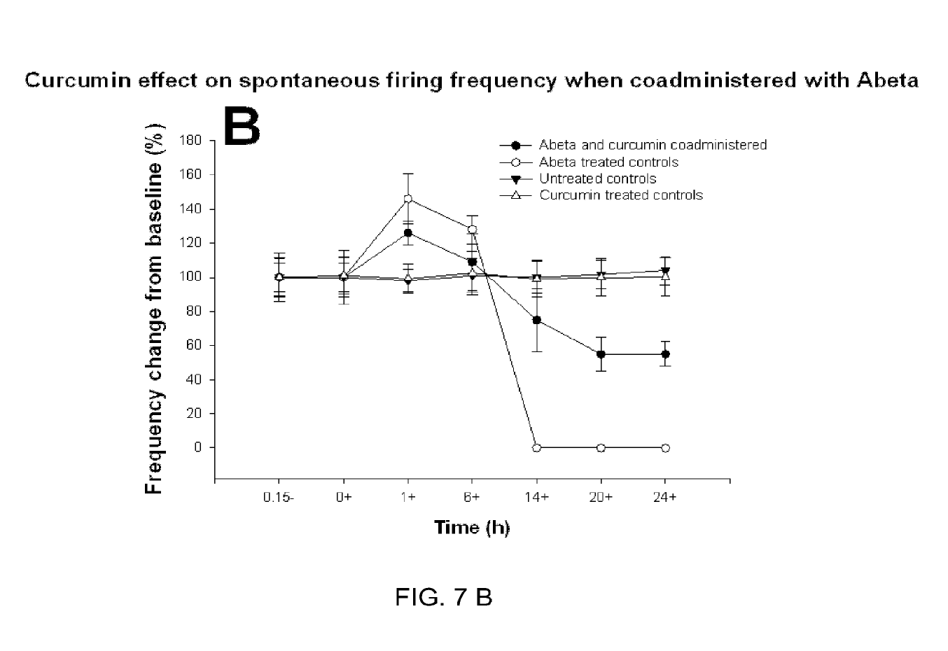

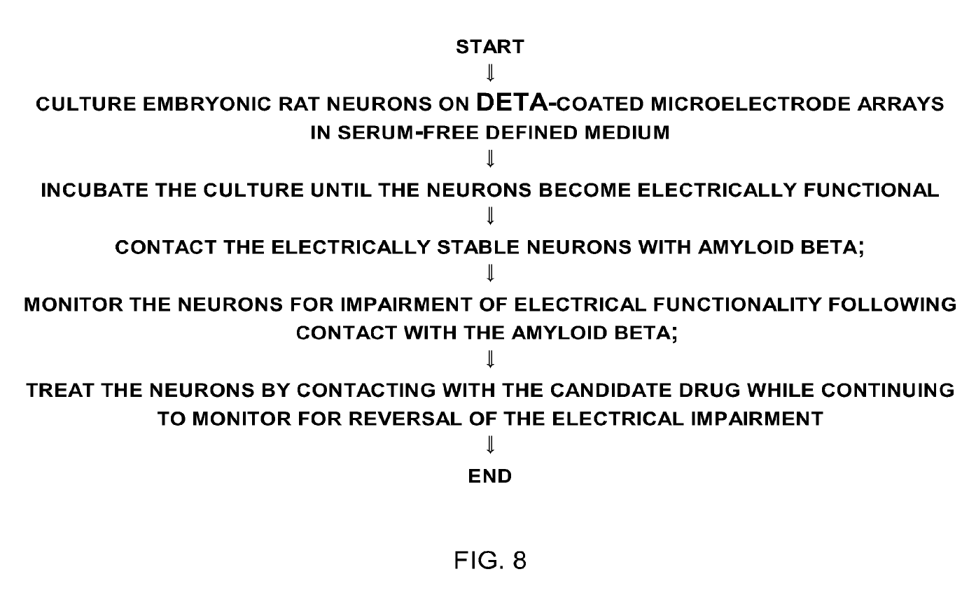

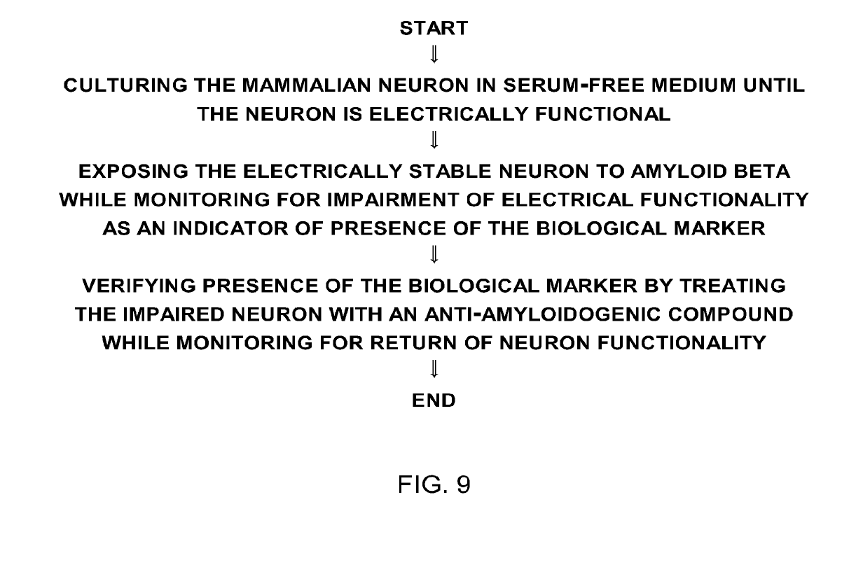

Thus, having demonstrated that Aβ functional toxicity could be reproduced using multielectrode arrays, a screening assay was then demonstrated by measuring the recovery of the lost functionality using the anti-amyloidogenic compound curcumin. Based on the control methodology in FIG. 6, two modes of curcumin application were used. Curcumin was coadministered with Aβ to the cells on the MEAs for a 24 hour period. In the second set of experiments curcumin was applied sequentially after the cells were exposed to Aβ for 24 hrs. We observed that functional recovery as recorded by the MEAs was similar to the patch-clamp experiments. As seen in FIG. 7A, when curcumin was coadministered with Aβ, the cells were able to maintain 54.960.7% (mean6SEM) of their baseline firing activity, as opposed to a complete loss of functionality when treated with Aβ alone. The decline in firing frequency was more gradual and the drop in firing frequency reached a plateau about 18 hrs after curcumin and Aβ were coadministered. Administration of curcumin after the cells were exposed to Aβ for 24 hrs resulted in a gradual recovery of firing frequency to 29.960.7% (mean6SEM) of the baseline (FIG. 7B). In this paradigm, recovery of spontaneous firing was observed around 10 hrs after curcumin was applied post Aβ exposure. The recovery of spontaneous firing frequency obtained with curcumin treatment was comparable to results obtained with patch clamp electrophysiology using similar experimental paradigms, as shown in FIG. 6.

### Discussion

Our initial results using whole-cell patch clamp electrophysiology demonstrated that Aβ affects electrical functionality earlier and at lower concentrations than which affect the survival of the cells. It is possible this effect could also precede synapse degradation or that it may be its upstream cause. Previous results had hinted at this idea, for example Chen and coworkers reported that various low concentrations of Aβ inhibited long-term potentiation (LTP) in hippocampal slices [2,38,39]. Based on these results, Ahuja et al. used MEA technology to measure Aβ effect on LTP in organotypic hippocampal cultures [40]. The importance of these investigations is highlighted by the significant need in the pharmaceutical industry for an in vitro model of the early stages of Alzheimer disease and the functional effects of Aβ on neurons observed in the study might be considered as an in vitro AD model.

We then utilized this result to create a high-throughput screening method for antagonists of this functional toxicity caused by Aβ. The MEAs made it possible to screen a significantly higher number of cells for Aβ and drug effects in a much shorter amount of time than patch-clamp electrophysiology would have required. Development of this method could have a high impact on drug development in Alzheimer's disease (AD). The molecular target of Aβ toxicity is not well known, thus this functional screen could result in novel effective compounds or therapeutic targets. We have shown that multielectrode arrays (MEAs) can be used to reliably detect functional effects of low doses of Aβ (100 nM) as well as screen for the rescue effect of curcumin. When applied to hippocampal neurons cultured on MEAs Aβ had a pronounced effect on the spontaneous firing of the cells, even at concentrations in the nanomolar range. Treatment with Aβ stopped spontaneous activity completely and the time for cessation was concentration dependent. The Aβ oligomerization inhibitor, curcumin, was able to partially reverse the loss of spontaneous activity. In accordance with our earlier patch clamp experiments, curcumin was more effective in inhibiting the effect of Aβ when it was coadministered with it as opposed to the experiments in which it was applied 24 hrs after Aβ exposure.

Interestingly, after Aβ exposure, there was a slight but consistent increase in firing frequency just before the decline of spontaneous activity. The initial increase in firing frequency we observed at all tested Aβ concentrations could be due to an earlier reported direct depolarizing effect of Aβ on the membrane potential or to the reputed ability of Aβ to enhance glutamate-mediated excitotoxicity [41,42] by its action on NMDA receptors and consequently, through an increased influx of Ca2+.

In comparison to slice preparation, our method, measurement of the effect of Aβ on spontaneous activity of cultured neurons, is significantly simpler and more applicable in high-throughput screen methodology. Another benefit of this MEA AD model, compared to our patch-clamp experiments, was that we were able to follow the time course of the action of curcumin on the Aβ modified activity of the same population of cells. When Aβ and curcumin were applied together, curcumin reduced the deleterious effect of Aβ without a significant change in the time course of Aβ action (FIG. 5A). When Aβ and curcumin were applied sequentially, curcumin reversed the effect of Aβ and helped the cells to partially recover their spontaneous firing activity (FIG. 5A). Curcumin was more effective when administered together with Aβ ; the cells were able to retain about 55% of their firing capability compared to untreated controls when coadministered as opposed to only 30% when sequentially administered. It has been shown that curcumin was able to inhibit Aβ oligomer formation and reduce amyloid toxicity in vitro [20]. In the presence of curcumin, reduced aggregation from monomeric Aβ and improved disassembly of preformed Aβ aggregates was observed [20]. Curcumin's ability to disassemble pre-formed Aβ aggregates could account for its protective effect against Aβ toxicity in the coadministration experiments, but the mechanism involved in the reversal of Aβ toxicity in the post-administration experiments needs further clarification.

In conclusion, our work demonstrates that it is possible to develop a high-throughput screen for the measurements of drug effects on functional toxicity of low concentrations of Aβ and this model might be considered as an in vitro functional model of the development of Alzheimer's disease. This screen method, based on MEA technology, which enables the screening of a large number of cells, and the study of pathogen and drug effects on the same population of cells over an extended period of time, could find important applications in pharmaceutical drug development and could lead to novel drug candidates or therapies for AD. Moreover, based on similar principles, MEA technology can be potentially extended to study in vitro models of other neurodegenerative diseases as well.

Accordingly, in the drawings and specification there have been disclosed typical preferred embodiments of the invention and although specific terms may have been employed, the terms are used in a descriptive sense only and not for purposes of limitation. The invention has been described in considerable detail with specific reference to these illustrated embodiments. It will be apparent, however, that various modifications and changes can be made within the spirit and scope of the invention as described in the foregoing specification and as defined in the appended claims.

### REFERENCES

1. Klein W L (2002) A beta toxicity in Alzheimer's disease: globular oligomers (ADDLs) as new vaccine and drug targets. Neurochemistry International 41(5): 345-352.
2. Lambert M P, Barlow A K, Chromy B A, Edwards C, Freed R, et al. (1998) Diffusible, nonfibrillar ligands derived from A beta(1-42) are potent central nervous system neurotoxins. Proceedings of the National Academy of Sciences of the United States of America 95(11): 6448-6453.
3. St George-Hyslop P H, Petit A (2005) Molecular biology and genetics of Alzheimer's disease. Comptes Rendus Biologies 328(2): 119-130.4. Townsend K P, Pratico D (2005) Novel therapeutic opportunities for Alzheimer's disease: focus on nonsteroidal anti-inflammatory drugs. Faseb Journal 19(12): 1592-1601.
5. Yankner B A (1996) Mechanisms of neuronal degeneration in Alzheimer's disease. Neuron 16(5): 921-932.
6. Hardy J, Selkoe D J (2002) Medicine—The amyloid hypothesis of Alzheimer's disease: Progress and problems on the road to therapeutics. Science 297(5580): 353-356.
7. Shankar G M, Li S, Mehta T H, Garcia-Munoz A, Shepardson N E, et al. (2008) Amyloid-[beta] protein dimers isolated directly from Alzheimer's brains impair synaptic plasticity and memory Nature Medicine 14(8): 837-842.
8. Knobloch M, Mansuy I M (2008) Dendritic spine loss and synaptic alterations in Alzheimer's disease. Molecular Neurobiology 37(1): 73-82.
9. Lacor P N (2007) Advances on the understanding of the origins of synaptic pathology in AD. Current Genomics 8(8): 486-508.
10. Lacor P N, Buniel M C, Furlow P W, Clemente A S, Velasco P T, et al. (2007) Neurobiology of Disease Aβ Oligomer-Induced Aberrations in Synapse Composition, Shape, and Density Provide a Molecular Basis for Loss of Connectivity in Alzheimer's Disease. Journal of Neuroscience Methods 27(4): 796-807.
11. Walsh D M, Selkoe D J (2007) Aβ Oligomers—a decade of discovery Journal of Neurochemistry 101: 1172-1184.
12. Francis P T (2008) Glutamatergic approaches to the treatment of cognitive and behavioural symptoms of Alzheimer's disease. Neurodegenerative Diseases 5(3-4): 241-243.
13. Koike T, Yang Y, Suzuki K, Zheng X (2008) Axon & dendrite degeneration: its mechanisms and protective experimental paradigms. Neurochem Internat 52(4-5): 751-760.
14. Waataja J J, Kim H J, Roloff A M, Thayer S A (2008) Excitotoxic loss of postsynaptic sites is distinct temporally and mechanistically from neuronal death Journal of Neurochemistry 104(2): 364-375.
15. Hua J Y, Smith S J (2004) Neural activity and the dynamics of central nervous system development. Nature Neuroscience 7: 327-332.
16. Nimmrich V, Grimm C, Draguhn A, Barghorn S, Lehmann A, et al. (2008) Amyloid b oligomers (A b (1-42) globulomer) suppress spontaneous synaptic activity by inhibition of P/Q-type calcium currents Journal of Neuroscience Methods 28(4): 788-797.
17. Hamaguchi T, Ono K, Yamada M (2006) Anti-amyloidogenic therapies: strategies for prevention and treatment of Alzheimer's disease. Cellular and Molecular Life Sciences 63(13): 1538-1552.
18. Zhao B L, Li X J, He R G, Cheng S J, Xin W J (1989) Scavenging Effect of Extracts of Green Tea and Natural Antioxidants on Active Oxygen Radicals. Cell Biophysics 14(2): 175-185.
19. Lim G P, Chu T, Yang F S, Beech W, Frautschy S A, et al. (2001) The curry spice curcumin reduces oxidative damage and amyloid pathology in an Alzheimer transgenic mouse. Journal of Neuroscience 21(21): 8370-8377.
20. Yang F S, Lim G P, Begum A N, Ubeda O J, Simmons M R, et al. (2005) Curcumin inhibits formation of amyloid beta oligomers and fibrils, binds plaques, and reduces amyloid in vivo. Journal of Biological Chemistry 280(7): 5892-5901.
21. Gureviciene I, Ikonen S, Gureviclus K, Sarkaki A, van Groen T, et al. (2004) Normal induction but accelerated decay of LTP in APP+PS1 transgenic mice. Neurobiology of Disease 15(2): 188-195.
22. Jhamandas J H, Cho C, Jassar B, Harris K, MacTavish D, et al. (2001) Cellular mechanisms for amyloid beta-protein activation of rat cholinergic basal forebrain neurons. Journal of Neurophysiology 86(3): 1312-1320.
23. Egert U, Schlosshauer B, Fennrich S, Nisch W, Fejtl M, et al. (1998) A novel organotypic long-term culture of the rat hippocampus on substrate-integrated multielectrode arrays. Brain Research Protocols 2(4): 229-242.
24. Jung D R, Cuttino D S, Pancrazio J J, Manos P, Cluster T, et al. (1998) Cell-based sensor microelectrode array characterized by imaging x-ray photoelectron spectroscopy, scanning electron microscopy, impedance measurements, and extracellular recordings. Journal of Vacuum Science & Technology a-Vacuum Surfaces and Films 16(3): 1183-1188.
25. Natarajan A, Molnar P, Sieverdes K, Jamshidi A, Hickman J J (2006) Microelectrode array recordings of cardiac action potentials as a high throughput method to evaluate pesticide toxicity. Toxicology in vitro 20(3): 375-381.
26. Offenhausser A, Sprossler C, Matsuzawa M, Knoll W (1997) Field-effect transistor array for monitoring electrical activity from mammalian neurons in culture. Biosensors & Bioelectronics 12(8): 819-826.
27. Nam Y, Wheeler B C, Heuschkel M O (2006) Neural recording adn stimulation of dissociated hippocampal cultures using microfabricated three-dimensional tip electrode array. Journal of Neuroscience Methods 155(2): 296-299.
28. Hofmann F, Bading H (2006) Long term recordings with microelectrode arrays: Studies of transcription-dependent neuronal plasticity and axonal regeneration. Journal of Physiology-Paris 99(2-3): 125-132.
29. Thomas C A, Springer P A, Okun L M, Berwaldn Y, Loeb G E (1972) Miniature Microelectrode Array to Monitor Bioelectric Activity of Cultured Cells. Experimental Cell Research 74(1): 61-&.
30. Gross G W, Gramowski A, Schiffmann D (1997) Neural network cultures on multielectrode chips: Self-organization of electrically active networks and their uses in neurotoxicology and neuropharmacology. European Journal of Cell Biology 74: 36-36.
31. Meyer T, Boven K H, Gunther E, Fejtl M (2004) Micro-electrode arrays in cardiac safety pharmacology—A novel tool to study QT interval prolongation. Drug Safety 27(11): 763-772.
32. Chang J C, Brewer G J, Wheeler B C (2001) Modulation of neural network activity by patterning. Biosensors & Bioelectronics 16(7-8): 527-533.
33. Golan H, Mikenberg K, Greenberger V, Segal M (2000) GABA withdrawal modifies network activity in cultured hippocampal neurons. Neural Plasticity 7(1-2): 31-42.
34. Gross G W, Rhoades B K, Azzazy H M E, Wu M C (1995) The Use of Neuronal Networks on Multielectrode Arrays as Biosensors. Biosensors & Bioelectronics 10(6-7): 553-567.
35. Gramowski A, Jugelt K, Stuwe S, Schulze R, McGregor G P, et al. (2006) Functional screening of traditional antidepressants with primary cortical neuronal networks grown on multielectrode neurochips. European Journal of Neuroscience 24(2): 455-465.
36. Keefer E W, Norton S J, Boyle N A J, Talesa V, Gross G W (2001) Acute toxicity screening of novel AChE inhibitors using neuronal networks on microelectrode arrays. Neurotoxicology 22(1): 3-12.
37. Parviz M, Gross G W (2007) Quantification of zinc toxicity using neuronal networks on microelectrode arrays. Neurotoxicology 28(3): 520-531.
38. Chen Q S, Kagan B L, Hirakura Y, Xie C W (2000) Impairment of hippocampal long-term potentiation by Alzheimer amyloid beta-peptides. Journal of Neuroscience Research 60(1): 65-72.
39. Wang H W, Pasternak J F, Kuo H, Ristic H, Lambert M P, et al. (2002) Soluble oligomers of beta amyloid (1-42) inhibit long-term potentiation but not longterm depression in rat dentate gyrus. Brain Research 924(2): 133-140.
40. Ahuja T K, Mielke J G, Comas T, Chakravarthy B, Mealing G A R (2007) Hippocampal slice cultures integrated with multi-electrode arrays: A model for study of long-term drug effects on synaptic activity. Drug Development Research 68(2): 84-93.
41. Lipton S A (2006) Paradigm shift in neuroprotection by NMDA receptor blockade: Memantine and beyond. Nature Reviews Drug Discovery 5(2): 160-170.
42. Mattson M P, Cheng B, Davis D, Bryant K, Lieberburg I, et al. (1992) Beta-Amyloid Peptides Destabilize Calcium Homeostasis and Render Human Cortical-Neurons Vulnerable to Excitotoxicity. Journal of Neuroscience 12(2): 376-389.
43. Schaffner A E, Barker J L, Stenger D A, Hickman J J (1995) Investigation of the factors necessary for growth of hippocampal neurons in a defined system. Journal of Neuroscience Methods 62(1-2): 111-119.
44. Das M, Molnar P, Devaraj H, Poeta M, Hickman J J (2003) Electrophysiological and morphological characterization of rat embryonic motoneurons in a defined system. Biotechnology Progress 19(6): 1756-1761.
45. Brewer G J, Torricelli J R, Evege E K, Price P J (1993) Optimized Survival of Hippocampal-Neurons in B27-Supplemented Neurobasal(™), a New Serum-Free Medium Combination. Journal of Neuroscience Research 35(5): 567-576.

That which is claimed:

1. A method of screening a candidate drug for effectiveness in reversing amyloid beta neurotoxicity, the method comprising:
culturing embryonic rat neurons on DETA-coated microelectrode arrays in serum-free defined medium;
incubating the culture until the neurons become electrically functional;
contacting the electrically stable neurons with amyloid beta;
monitoring the neurons for impairment of electrical functionality following contact with the amyloid beta; and
treating the neurons by contacting with the candidate drug while continuing to monitor for reversal of the electrical impairment.

2. A method of screening a compound for effectiveness in treating amyloid beta neurotoxicity, the method comprising:
culturing mammalian neurons in serum-free defined medium until the neurons are electrically functional;
exposing the electrically stable neurons to amyloid beta;
monitoring the exposed neurons for impairment of electrical functionality; and
treating the exposed neurons with the candidate drug while monitoring their electrical activity for reversal of impairment.

3. A method of identifying a mammalian neuron having a biological marker conferring predisposition to development of Alzheimer's disease, the method comprising:
culturing the mammalian neuron in serum-free medium until the neuron is electrically functional;
exposing the electrically stable neuron to amyloid beta while monitoring for impairment of electrical functionality as an indicator of presence of said biological marker; and
verifying presence of the biological marker by treating the impaired neuron with an anti-amyloidogenic compound while monitoring for return of neuron functionality.
